(19) United States
(12) Patent Application Publication
(10) Pub. No.: US 2025/0161707 A1
(43) Pub. Date: May 22, 2025

(54) NEGATIVE PRESSURE LASER HANDPIECE

(71) Applicant: The General Hospital Corporation, Boston, MA (US)

(72) Inventor: Richard R. Anderson, Boston, MA (US)

(21) Appl. No.: 18/870,158

(22) PCT Filed: May 26, 2023
(86) PCT No.: PCT/US2023/067560

§ 371 Date: Nov. 27, 2024

Related U.S. Application Data
(60) Provisional application No. 63/346,789, filed on May 27, 2022.

(51) Int. Cl. A61N 5/06

(52) U.S. Cl. CPC A61N 5/0616

(57) ABSTRACT

Disclosed are devices and methods for treating a skin condition (e.g., cutaneous neurofibroma tumors) of a subject. The device comprises: a chamber having an end wall, a side wall extending away from the end wall, and an opening opposite the end wall, wherein the side wall terminates in a contact surface for sealing against skin of a subject; a vacuum source in fluid communication with the interior space of the chamber, the vacuum source being structured to create negative pressure in the chamber; and a light source for irradiating a region of the skin of the subject with light, the light source being selected from the group consisting of laser light sources and wavelength-filtered light sources, the light source being positioned to direct the light at the region of the skin of the subject when the light source is activated to treat the skin condition.

### CROSS-REFERENCE TO RELATED APPLICATIONS

This application is based on, claims benefit of, and claims priority to U.S. Application No. 63/346,789, filed on May 27, 2022, which is hereby incorporated by reference herein in its entirety for all purposes.

### STATEMENT REGARDING FEDERALLY SPONSORED RESEARCH

Not Applicable.

### BACKGROUND OF THE INVENTION

### 1. Field of the Invention

The invention relates to devices and methods for treating a skin condition (e.g., cutaneous neurofibroma tumors) of a subject.

### 2. Background and Description of the Related Art

Neurofibromatosis type I (NF-1) is the most common genetic disease of mankind, arising from mutations in a large gene called neurofibromin. Half the cases are familial, and half are spontaneous. The disease causes nerve, brain, eye and skin problems due to tumors called neurofibromas. These so-called “benign” skin tumors begin in childhood but are invisible, then progressively grow during adolescence and adulthood. As children, people with NF-1 appear normal. However by adulthood, most NF-1 patients have hundreds to thousands of obvious cutaneous neurofibromas (cNF), some of which are painful, requiring surgery, and very disfiguring.

Previous treatments for cNF are surgical removal or destruction when the tumors become numerous and large enough, usually done under general anesthesia at great expense, leaving wounds and scars. Neurologists and dermatologists just do not have a good treatment for the skin tumors. Gross disfigurement is the number one quality-of-life issue for people with NF. After looking normal as children, they face progressive loss of self-image, embarrassment, bullying, social isolation, difficulty working in a public job, shame, major depression and suicidality.

Clearly, what is needed therefore is a practical way to treat cNF tumors (and other skin neoplasms) when they are small and barely visible, keeping people with NF-1 looking normal throughout their life. There is also a need to treat skin malformations when they are small and barely visible.

### SUMMARY OF THE INVENTION

To address these limitations, the present disclosure provides devices and methods for treating skin neoplasms (such as cutaneous neurofibroma tumors) and skin malformations of a subject.

In one aspect, the present disclosure provides a device for treating a condition of skin of a subject. The device comprises: (i) a chamber having an end wall, a side wall extending away from the end wall, and an opening opposite the end wall, wherein the end wall, the side wall and the opening define an interior space of the chamber, wherein the end wall comprises a transparent window, wherein the side wall terminates in a contact surface for sealing against skin of a subject, wherein the contact surface defines the opening, wherein the side wall has a longitudinal length such that the end wall is spaced greater than ten millimeters from the skin of the subject when the contact surface seals against skin of the subject; (ii) a vacuum source in fluid communication with the interior space of the chamber, the vacuum source being structured to create negative pressure in the chamber; and (iii) a light source for irradiating a region of the skin of the subject with light, the light source being selected from the group consisting of laser light sources and wavelength-filtered light sources, the light source being positioned to direct the light through the transparent window at the region of the skin of the subject when the light source is activated.

In yet another aspect, the present disclosure provides a device for treating a condition of skin of a subject. The device comprises: (i) a chamber having an end wall, a side wall extending away from the end wall, and an opening opposite the end wall, wherein the end wall, the side wall and the opening define an interior space of the chamber, wherein the end wall comprises a transparent window, wherein the side wall terminates in a contact surface for sealing against skin of a subject, wherein the contact surface defines the opening; (ii) a vacuum source in fluid communication with the interior space of the chamber, the vacuum source being structured to create negative pressure in the chamber; (iii) a pressure regulator operable to control a suction level of the vacuum source such that when the contact surface seals against the skin of the subject, the negative pressure created in the chamber by the vacuum source does not draw the skin of the subject into contact with the end wall of the chamber; and (iv) a light source for irradiating a region of the skin of the subject with light, the light source being selected from the group consisting of laser light sources and wavelength-filtered light sources, the light source being positioned to direct the light through the transparent window at the region of the skin of the subject when the light source is activated.

In still another aspect, the present disclosure provides a method for treatment of a skin neoplasm of skin of a subject. The method can comprise: (a) providing a chamber having an end wall, a side wall extending away from the end wall, and an opening opposite the end wall, wherein the end wall, the side wall and the opening define an interior space of the chamber, wherein the side wall terminates in a contact surface for sealing against skin of a subject, wherein the contact surface defines the opening; (b) positioning the contact surface of the chamber on the skin of the subject such that the contact surface surrounds the skin neoplasm and the contact surface seals against the skin of the subject; (c) creating negative pressure in the interior space of the chamber; and (d) irradiating the skin neoplasm with light from a light source selected from the group consisting of laser light sources and wavelength-filtered light sources.

In yet another aspect, the present disclosure provides a method for treatment of a skin malformation of skin of a subject. The method can comprise: (a) providing a chamber having an end wall, a side wall extending away from the end wall, and an opening opposite the end wall, wherein the end wall, the side wall and the opening define an interior space of the chamber, wherein the side wall terminates in a contact surface for sealing against skin of a subject, wherein the contact surface defines the opening; (b) positioning the contact surface of the chamber on the skin of the subject such that the contact surface surrounds the skin malformation and the contact surface seals against the skin of the subject; (c) creating negative pressure in the interior space of the chamber; and (d) irradiating the skin malformation with light from a light source selected from the group consisting of laser light sources and wavelength-filtered light sources.

These and other features, aspects and advantages of various embodiments of the present disclosure will become better understood with regard to the following description, appended claims, and accompanying Figures.

### BRIEF DESCRIPTION OF THE DRAWINGS

FIG. 1 is a perspective view of a device for treating a skin condition (e.g., cutaneous neurofibroma tumors) according to aspects of the present disclosure.

FIG. 2 shows neurofibroma tumor response to treatment using a non-limiting example device of the present disclosure, in a person with neurofibromatosis type I (NF-1). Panel (A) shows a skin region prior to treatment. Panel (B) shows immediate response after treatment with a negative-pressure assisted alexandrite laser at 755 nm. 3 milliseconds, 100 J/cm², 8 mm spot size exposure, showing darkening due to photocoagulation of tumor target blood vessels; treatment of all five tumors took approximately 1 minute. Panel (C) shows the pre-treatment photo with the five treated tumors marked (white circles). Panel (D) shows elimination (in two) and substantial reduction (in three) of all treated tumors, 3 months after treatment, without scarring or other side effects.

FIG. 3 shows a microscopy (H&E stain) of a human neurofibroma tumor after treatment with a prototype negative-pressure device and alexandrite laser system. A fluence of 100 J/cm² was used, with an 8 mm diameter exposure spot at 755 nm wavelength, and 3 ms pulse duration, with a pressure of ˜125 mmHg (about ⅙ of an atmosphere, a negative pressure of about ⅚ atmosphere) within the negative pressure chamber. Immediately after treatment, throughout the tumor blood vessels are distended and thermally damaged, filled with non-flowing thermally-damaged blood. Three months after treatment, there is loss of the tumor and in the example shown in Figure, a small residual tumor remains after one treatment.

### DETAILED DESCRIPTION OF THE INVENTION

Before the present invention is described in further detail, it is to be understood that the invention is not limited to the particular embodiments described. It is also to be understood that the terminology used herein is for the purpose of describing particular embodiments only and is not intended to be limiting. The scope of the present invention will be limited only by the claims. As used herein, the singular forms “a”, “an”, and “the” include plural embodiments unless the context clearly dictates otherwise.

It will be appreciated by those skilled in the art that while the disclosed subject matter is described herein in connection with particular embodiments and examples, the invention is not necessarily so limited, and that numerous other embodiments, examples, uses, modifications and departures from the embodiments, examples and uses are intended to be encompassed by the claims attached hereto.

It should be apparent to those skilled in the art that many additional modifications beside those already described are possible without departing from the inventive concepts. In interpreting this disclosure, all terms should be interpreted in the broadest possible manner consistent with the context. Variations of the term “comprising”, “including”, or “having” should be interpreted as referring to elements, components, or steps in a non-exclusive manner, so the referenced elements, components, or steps may be combined with other elements, components, or steps that are not expressly referenced. Embodiments referenced as “comprising”, “including”, or “having” certain elements are also contemplated as “consisting essentially of” and “consisting of” those elements, unless the context clearly dictates otherwise.

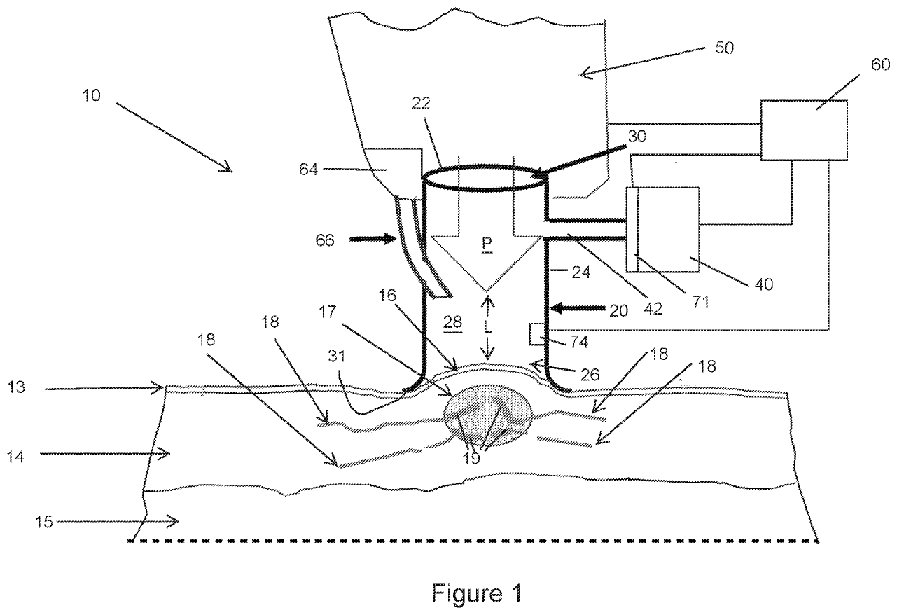

Looking now at FIG. 1, there is shown a non-limiting example device 10 according to the invention for treating a condition (e.g. a skin neoplasm or a skin malformation) of skin of a subject (e.g., a mammal such as a human). The subject has epidermis 13, dermis 14, subcutaneous fat 15, and a region 16 near a target tumor 17 with distended vessels 19, and blood vessels 18 supplying the target tumor 17.

The device 10 includes a chamber 20 having an end wall 22, a side wall 24 extending away from the end wall 22, and an opening 26 opposite the end wall 22. The end wall 22, the side wall 24, and the opening 26 define an interior space 28 of the chamber 20. The end wall 22 comprises a transparent window 30. The side wall 24 terminates in a contact surface 31 for sealing against the skin (i.e., epidermis 13) of the subject. The contact surface 31 defines the opening 26. The side wall 24 has a longitudinal length along axis L such that the end wall 22 is spaced greater than ten millimeters from the skin (i.e., epidermis 13) of the subject when the contact surface 31 seals against skin (i.e., epidermis 13) of the subject. In the device 10, the side wall 24 of the chamber 20 is cylindrical and has a diameter in a range of 10 to 50 millimeters, and the side wall 24 has a longitudinal length along axis L of greater than 10 up to 100 millimeters. The end wall 22 and the side wall 24 of the chamber 20 can be transparent.

In another embodiment, the end wall 22 is spaced greater than twenty millimeters from the skin (i.e., epidermis 13) of the subject when the contact surface 31 seals against skin (i.e., epidermis 13) of the subject. In another embodiment, the end wall 22 is spaced greater than thirty millimeters from the skin (i.e., epidermis 13) of the subject when the contact surface 31 seals against skin (i.e., epidermis 13) of the subject. In another embodiment, the end wall 22 is spaced greater than forty millimeters from the skin (i.e., epidermis 13) of the subject when the contact surface 31 seals against skin (i.e., epidermis 13) of the subject. In another embodiment, the chamber is breathable.

The device 10 includes a vacuum source 40 in fluid communication with the interior space 28 of the chamber 20. The vacuum source 40 is structured to create negative pressure in the chamber 20. “Negative pressure” means pressure negative relative to atmospheric pressure.

The device 10 includes a light source 50 for irradiating the region 16 of the skin (i.e., epidermis 13) of the subject with light along light beam path P. The light source 50 can be laser light source or a wavelength-filtered light source. The light source 50 is positioned to direct the light through the transparent window 30 along light beam path P at the region 16 of the skin of the subject when the light source is activated. The light source 50 can emit wavelengths that are preferentially absorbed by hemoglobin and/or oxyhemoglobin. In some embodiments, the light source emits wavelengths in a range of wavelengths from 500 nanometers to 2000 nanometers. In some embodiments, the light source emits wavelengths in a range of wavelengths from 500 nanometers to 1200 nanometers. In some embodiments, the light source emits wavelengths in a range of wavelengths from 700 nanometers to 800 nanometers. In some embodiments, the light source is an intense-pulsed-light source. In some embodiments, a spot size of the light has a diameter in a range of 1 to 20 millimeters, or 1 to 10 millimeters.

The device 10 includes a controller 60 which may comprise a microprocessor under the control of a software program stored in the controller memory. The controller 60 is in electrical communication with a coolant source 64 that provides coolant to the interior space 28 of the chamber 20 via conduit 66. The optional coolant source 64 can be a dynamic cryogen spray cooling port. The controller 60 is also in electrical communication with a pressure regulator 71 that is operable to control a suction level of the vacuum source 40. The pressure regulator is operable to control a suction level of the vacuum source 40 such that when the contact surface 31 seals against the skin of the subject, the negative pressure created in the chamber 20 by the vacuum source 40 does not draw the skin of the subject into contact with the end wall 22 comprising the transparent window 30 of the chamber 20. The controller 60 is also in electrical communication with a pressure sensor 74 in the chamber 20. Pressure, temperature, and so forth can be monitored by the controller 60.

The controller 60 can be configured to execute a program stored in the controller to: (i) activate the vacuum source 40 to create the negative pressure in the chamber 20, and (ii) irradiate the region 16 of the skin with light for a period of time such that the light is provided at a radiant exposure of at least 5 J/cm². The controller 60 can be configured to execute a program stored in the controller to: (ii) irradiate the region 16 of the skin with light for the period of time such that the light is provided at a radiant exposure of at least 50 J/cm², or at least 100 J/cm², or at least 150 J/cm². The radiant exposure (also referred to as fluence or dose) can be calculated with the following equation (Radiant exposure (J/cm²)=Irradiance (W/cm²)×Exposure time(s)). The controller 60 can be configured to execute a program stored in the controller to: (ii) irradiate the region of the skin with pulsed light at a pulse duration of 1 to 10 milliseconds.

The controller 60 can execute the program stored in the controller to: (iii) receive electrical signals from the pressure sensor 74 and continue activation of the vacuum source 40 for a time duration based on electrical signals received from the pressure sensor 74. In some embodiments, the time duration is greater than 1 second. In some embodiments, the time duration is greater than 10 seconds. In some embodiments, the time duration is greater than 20 seconds.

The controller 60 can execute the program stored in the controller to: (iv) continue activation of the vacuum source 40 for the time duration until the electrical signals received from the pressure sensor 74 indicate that negative pressure created in the chamber 20 has reached a predetermined pressure level. The controller 60 executes the program stored in the controller to: (iii) receive electrical signals from the pressure sensor 74 and continue activation of the pressure regulator 71 for a time duration based on electrical signals received from the pressure sensor 74. The controller 60 can execute the program stored in the controller to: (iv) continue activation of the pressure regulator 71 for the time duration until the electrical signals received from the pressure sensor 74 indicate that negative pressure created in the chamber 20 has reached a predetermined pressure level, such as any negative pressure level between about −10 mm Hg and −760 mm Hg, or between −50 mm Hg and −400 mm Hg. or between −100 mm Hg and −200 mm Hg. The blood that is drawn by negative pressure into the target vessels 19 is venous blood. Arteries are already under high internal pressure, and it would be very difficult to use negative pressure to distend an artery. In contrast, veins are thin-walled vessels with a low internal pressure; they can distend a lot with this invention. The hydrostatic pressure in veins is low, typically less than 100 mmHg and often just 5-10 mmHg. So suction greatly affects their blood content. This invention greatly distends veins; the neurofibromas which are usually normal-skin-color, become visibly bright red when suction is applied.

In one non-limiting example embodiment of a device according to the invention, the negative pressure device can be applied over a dermal tumor, which may be benign or malignant. The negative pressure chamber can be cylindrical, 1-5 cm in diameter, and 1-10 cm long. The laser can be, without limitation, a Candela alexandrite laser operated at 100 J/cm² fluence, 3 ms pulse duration, 755 nm wavelength to provide a uniform beam 8 mm in diameter at the skin surface. The edge which contacts skin forms a nominal seal with the skin surrounding the target tumor, and is shaped to minimize compression of the skin and blood vessels, with minimal contact force such that blood flow is allowed to occur from surrounding skin into the target tumor, engorging tumor vessels as shown in FIG. 1. Similar devices within the invention that use negative pressure applied to engorge target blood vessels, can be used in other organs either directly, endoscopically, with optical fiber light delivery, with minimally invasive access, or with open surgical access. The target vessels may be supplying blood to a target tumor, or may be a primary vascular malformation or vascular tumor. The medium within the negative pressure chamber in this example is air or other gas, but may be a liquid such as water or normal saline. In this example, negative pressure induces within the dermis of skin a pressure gradient that draws blood into the tumor, shown schematically in FIG. 1 with some exemplary blood vessels that become distended with blood within the tumor. A laser or other optical device provides one or more pulses of electromagnetic radiation at wavelength(s) absorbed by hemoglobins within the distended blood vessels of the target tumor, and which adequately penetrate within the tumor, at fluence(s) and pulse duration(s) capable of photocoagulation of the distended blood vessels. Examples of such lasers without limitation include frequency-doubled Nd: YAG or Nd fiber lasers (˜532 nm), pulsed dye lasers (˜590 nm), alexandrite (˜755 nm), Nd: YAG (˜1064 nm), diode (˜750-1200 nm), and various fiber (˜700-1200 nm) lasers. See examples of suitable lasers in Table 1 below.

TABLE 1

| | | |
|---|---|---|
| Laser | Wavelength(s) | Approx. Fluence (J/cm²) |

| | | | |
|---|---|---|---|
| KTP (various) | 532 | nm | 5-20 |
| Pulsed dye | 570-600 | nm | 5-20 |
| Alexandrite | 750-770 | nm | 50-150 |
| Diode | 750-1200 | nm | 50-300 |
| Nd (various) | 1060-1080 | nm | 150-400 |

A method according to the invention can be used for treatment of a skin neoplasm of skin of a subject. The method can comprise: (a) providing a chamber having an end wall, a side wall extending away from the end wall, and an opening opposite the end wall, wherein the end wall, the side wall and the opening define an interior space of the chamber, wherein the side wall terminates in a contact surface for sealing against skin of a subject, wherein the contact surface defines the opening; (b) positioning the contact surface of the chamber on the skin of the subject such that the contact surface surrounds the skin neoplasm and the contact surface seals against the skin of the subject; (c) creating negative pressure in the interior space of the chamber; and (d) irradiating the skin neoplasm with light from a light source selected from the group consisting of laser light sources and wavelength-filtered light sources. The skin neoplasm can be selected from the group consisting of skin tumors (including non-melanoma skin cancers and benign skin tumors) and warts (especially periungual and plantar warts). The skin neoplasm can be selected from the group consisting of neurofibroma tumors and basal cell carcinoma tumors. The skin neoplasm can be a cutaneous neurofibroma tumor.

Another method according to the invention can be used for treatment of a skin malformation of skin of a subject. The method can comprise: (a) providing a chamber having an end wall, a side wall extending away from the end wall, and an opening opposite the end wall, wherein the end wall, the side wall and the opening define an interior space of the chamber, wherein the side wall terminates in a contact surface for sealing against skin of a subject, wherein the contact surface defines the opening; (b) positioning the contact surface of the chamber on the skin of the subject such that the contact surface surrounds the skin malformation and the contact surface seals against the skin of the subject; (c) creating negative pressure in the interior space of the chamber; and (d) irradiating the skin malformation with light from a light source selected from the group consisting of laser light sources and wavelength-filtered light sources. The skin malformation can be selected from the group consisting of vascular lesions. The skin malformation can be a port-wine stain. The skin malformation can be telangiectasia.

In one example embodiment of a method for treatment of a skin neoplasm or a skin malformation of skin of a subject according to the invention, the light source emits wavelengths that are preferentially absorbed by hemoglobin and/or oxyhemoglobin. In one example embodiment of a method for treatment of a skin neoplasm or a skin malformation of skin of a subject according to the invention, the light source emits wavelengths in a range of wavelengths from 500 nanometers to 2000 nanometers. In one example embodiment of a method for treatment of a skin neoplasm or a skin malformation of skin of a subject according to the invention, the light source emits wavelengths in a range of wavelengths from 500 nanometers to 1200 nanometers. In one example embodiment of a method for treatment of a skin neoplasm or a skin malformation of skin of a subject according to the invention, the light source emits wavelengths in a range of wavelengths from 700 nanometers to 800 nanometers.

In one example embodiment of a method for treatment of a skin neoplasm or a skin malformation of skin of a subject according to the invention, the light source is a laser light source. In one example embodiment of a method for treatment of a skin neoplasm or a skin malformation of skin of a subject according to the invention, the light source is an intense-pulsed-light source. In one example embodiment of a method for treatment of a skin neoplasm or a skin malformation of skin of a subject according to the invention, the light source is a wavelength-filtered light source. In one example embodiment of a method for treatment of a skin neoplasm or a skin malformation of skin of a subject according to the invention, a spot size of the light has a diameter in a range of 1 to 20 millimeters, 1 to 10 millimeters.

In one example embodiment of a method for treatment of a skin neoplasm or a skin malformation of skin of a subject according to the invention, the side wall of the chamber has a longitudinal length such that the end wall is spaced greater than ten millimeters from the skin of the subject when the contact surface seals against skin of the subject. In one example embodiment of a method for treatment of a skin neoplasm or a skin malformation of skin of a subject according to the invention, the side wall of the chamber has a longitudinal length such that the end wall is spaced greater than twenty millimeters from the skin of the subject when the contact surface seals against skin of the subject. In one example embodiment of a method for treatment of a skin neoplasm or a skin malformation of skin of a subject according to the invention, the side wall of the chamber has a longitudinal length such that the end wall is spaced greater than thirty millimeters from the skin of the subject when the contact surface seals against skin of the subject. In one example embodiment of a method for treatment of a skin neoplasm or a skin malformation of skin of a subject according to the invention, the side wall of the chamber has a longitudinal length such that the end wall is spaced greater than forty millimeters from the skin of the subject when the contact surface seals against skin of the subject. In one example embodiment of a method for treatment of a skin neoplasm or a skin malformation of skin of a subject according to the invention, the chamber is breathable.

In one example embodiment of a method for treatment of a skin neoplasm or a skin malformation of skin of a subject according to the invention, step (d) comprises irradiating the skin neoplasm or the skin malformation with light from the light source such that the light is provided at a radiant exposure of at least 5 J/cm². In one example embodiment of a method for treatment of a skin neoplasm or a skin malformation of skin of a subject according to the invention, step (d) comprises irradiating the skin neoplasm or the skin malformation with light from the light source such that the light is provided at a radiant exposure of at least 50 J/cm². In one example embodiment of a method for treatment of a skin neoplasm or a skin malformation of skin of a subject according to the invention, step (d) comprises irradiating the skin neoplasm or the skin malformation with light from the light source such that the light is provided at a radiant exposure of at least 100 J/cm². In one example embodiment of a method for treatment of a skin neoplasm or a skin malformation of skin of a subject according to the invention, step (d) comprises irradiating the skin neoplasm or the skin malformation with pulsed light at a pulse duration of 1 to 10 milliseconds.

In one example embodiment of a method for treatment of a skin neoplasm or a skin malformation of skin of a subject according to the invention, step (c) comprises creating negative pressure in the interior space of the chamber for a time duration greater than 1 second. In one example embodiment of a method for treatment of a skin neoplasm or a skin malformation of skin of a subject according to the invention, step (c) comprises creating negative pressure in the interior space of the chamber for a time duration greater than 10 seconds. In one example embodiment of a method for treatment of a skin neoplasm or a skin malformation of skin of a subject according to the invention, step (c) comprises creating negative pressure in the interior space of the chamber for a time duration greater than 20 seconds. The negative pressure can be between about −10 and −760 mm Hg, or between −50 mm Hg and −400 mm Hg, or between −100 mm Hg and −200 mm Hg.

In one example embodiment of a method for treatment of a skin neoplasm or a skin malformation of skin of a subject according to the invention, step (d) comprises irradiating the skin neoplasm or the skin malformation with pulsed light from a laser light source such that the light is provided at a radiant exposure of at least 100 J/cm², the laser light source emits wavelengths in a range of wavelengths from 700 nanometers to 800 nanometers, the pulsed light has a pulse duration of 1 to 10 milliseconds, and a spot size of the light has a diameter in a range of 1 to 20 millimeters.

This invention comprises devices and methods that allow selective, effective treatment for removal of skin lesions including non-melanoma skin cancers, benign skin tumors such as neurofibromas, and some vascular lesions, by enhancing laser-induced irreversible damage of blood vessels that supply the skin lesions. Specifically, negative pressure is first used to increase the caliber of cutaneous blood vessels, and then a laser or intense-pulsed-light source emitting wavelength(s) that are preferentially absorbed by blood is used to cause blood-vessel-specific selective photothermolysis. This invention is particularly beneficial at treatment of the skin tumors of neurofibromatosis, a genetic disease that causes many soft, squishy tumors to grow in the skin. Because the tumors are soft, negative pressure distends blood vessels within the tumor more easily that those of the surrounding skin. Negative pressure thereby enhances the amount of target chromophores (hemoglobin and oxyhemoglobin) that enable treatment to preferentially damage the vessels supplying the tumor.

The tumors are infarcted (die from lack of blood supply), and are also preferentially heated, causing some irreversible thermal damage. More broadly, this invention can also be used to enhance treatment of other vascular lesions including microvascular malformations such as port wine stains. Other embodiments can include devices to produce and to regulate negative pressure, including application of negative pressure between −10 and −760 mm Hg. Furthermore, the negative pressure may be regulated to achieve a desired endpoint of color change in the target lesions, which may be detected by optical reflectance or by simple inspection.

This invention includes a method for selective killing and subsequent removal of a target tumor(s) by infarction. One example embodiment of a method according to the invention includes the steps of: (a) positioning a chamber device over a target tumor, (b) contacting the skin surface with an opening of the chamber such that skin surface makes a nominal seal with the chamber, (c) providing negative pressure between 10-760 mmHg in the chamber, (d) waiting typically 1-10 seconds for blood flowing into the tumor from surrounding tissue to distend blood vessels within the tumor, (e) exposing the tumor and nearby surrounding tissue to a pulse(s) of optical radiation at a wavelength(s) preferentially absorbed by blood and capable of penetration within the tumor and at a fluence and pulse duration that produces an immediately visible darkening of the tumor. The distention of target blood vessels renders them a more effective target for selective photothermolysis, by increasing their physical size, blood content, and by stretching the vessel walls which makes them thinner and more susceptible to thermal damage. After optical exposure, the plasma proteins and cells in blood are thermally denatured, and the blood no longer flows. The endothelial cells lining the vessels are also damaged, triggering a clotting process of whatever blood may again enter the vessels. Blood flow is stopped, and the vessel walls are irreversibly damaged. Cells in the tumor then die due to infarction (loss of blood supply), and hypoxia (loss of an oxygen supply) leading to elimination of the tumor during the subsequent approximately three months after treatment.

### Example

The following Example is provided to demonstrate and further illustrate certain embodiments and aspects of the present invention and is not to be construed as limiting the scope of the invention. The statements provided in the Example are presented without being bound by theory.

This Example reports results of a study of a prospective, direct comparison of four methods of non-invasive treatment of cutaneous neurofibromas (cNF). The purpose was to determine if minimally-invasive, rapid treatment(s) could eliminate small cNF when they first appear such that disfigurement could be prevented.

I became interested in helping children and young adults with neurofibromatosis type I (NF-1). NF-1 is the most common genetic disease of mankind, arising from mutations in a large gene called neurofibromin. Half the cases are familial, and half are spontaneous. The disease causes nerve, brain, eye and skin problems due to tumors called neurofibromas. These so-called “benign” skin tumors begin in childhood but are invisible, then progressively grow during adolescence and adulthood. As children, people with NF-1 appear normal. However by adulthood, most NF-1 patients have hundreds to thousands of obvious cutaneous neurofibromas (cNF), some of which are painful, requiring surgery, and very disfiguring.

Previous treatments for cNF are surgical removal or destruction when the tumors become numerous and large enough, usually done under general anesthesia at great expense, leaving wounds and scars. Neurologists and dermatologists just did not have a good treatment for the skin tumors. Gross disfigurement is the number one quality-of-life issue for people with NF. After looking normal as children, they face progressive loss of self-image, embarrassment, bullying, social isolation, difficulty working in a public job, shame, major depression and suicidality. Clearly, what is needed is a practical way to treat cNF tumors when they are small and barely visible, keeping people with NF-1 looking normal throughout their life. I questioned whether it was possible to inhibit these tumors with a local, non-invasive, rapid, well-tolerated, affordable treatment that leaves normal skin.

A classic and highly effective application of lasers in dermatology is the selective targeting of blood vessels for treatment of numerous vascular malformations and vascular tumors. Lasers or intense pulsed light (IPL) sources emitting wavelengths that are preferentially absorbed by hemoglobin and/or oxyhemoglobin are used to preferentially heat and destroy blood vessels. Pulses of light are used to cause selective photothermolysis (SP) of the vessels, i.e., to produced thermal injury that is localized to the target vessels without widespread injury to the surrounding tissue. This is a very powerful strategy. SP is now used for about 1 million treatments/month in dermatology, ophthalmology and laryngology to treat a variety of vascular targets. Despite its long and successful history, SP has not been fully developed.

I started working on physical and chemical ways to destroy neurofibromas. In the lab, I studied freshly-excised cNF tumors, obtained with permission after surgical removal, to identify optical properties of the tumors that might allow me to selectively target the tumors using light. Disappointingly, there was very little difference in optical properties between cNF and the normal surrounding dermis. However, excised tumors have no blood in them, so I thought that perhaps a vascular-targeting laser could still work. On histology, I noticed that all of the cNF tumors have a “cap” of normal skin overlying them (worth sparing). I also noticed a higher density of small blood vessels within most of the tumors compared with surrounding normal dermis, and located mainly near the margins of the tumors. Presumably, cNF tumors recruit host blood vessels via angiogenesis, as most solid tissue tumors do. Next, I cautiously treated small cNF tumors in a handful of adult patients with NF-1 on a compassionate basis, using FDA-approved devices off-label that might be effective. One of those was an alexandrite laser emitting at 755 nm wavelength, capable of deep vascular targeting. In a few tumors there was a selective effect, but in most tumors there was not.

I asked why was there an erratic, hit-or-miss, targeting of cNF by this powerful vascular laser? Variable blood content in cNF tumors was probably the cause. Then, it occurred to me that the blood content of a cNF tumor could be increased by application of negative pressure, and I reasoned furthermore that these tumors in particular would be susceptible because of their mechanical properties. The cNF tumors are very soft, pliable, doughy and easily deformed compared with normal skin, which by comparison is stiff. The small vessels in skin have no valves, so application of gentle suction (negative pressure) causes the vessels to fill with blood. I reasoned that the blood vessels in a cNF tumor would be more prone to distention, due to the soft elasticity of these tumors. Also, the presence of vessels at the tumor periphery suggested that a vascular targeting laser could effectively infarct the tumor. Applying suction of about 0.5 atm through a 12 mm diameter plastic tube applied to the skin surface, I noticed that cNF tumors clearly turn red and mildly distend several seconds after application of the suction. In my lab, I had a simple modification to an alexandrite laser handpiece built. Testing that on a few patients with NF-1, the variability of cNF response was less, and more importantly there was clear evidence of selective effect on the cNF tumors, noted by immediate darkening of the tumors without change in the adjacent skin. The darkening is due formation of methemoglobin, a product of thermal coagulation of blood. A brief exploration of dosimetry showed that a fluence of 80-100 J/cm² using a 3 ms, 8 mm diameter, 755 nm alexandrite laser beam reliably targeted the tumors.

Next, I launched a trial of cNF treatment using the Methods described below, comparing 4 different modalities—the vascular-specific 755 nm alexandrite laser with prototype suction handpiece, a tissue coagulating diode laser at 980 nm, an insulated radiofrequency needle, and local injection of deoxycholic acid (Kybella® from Abbvie Pharmaceuticals), a naturally occurring surfactant used for destruction of unwanted fat. From the study, it was already clear that the alexandrite laser with suction is effective for clearing small cNF. The tumors regress within about 90 days, leaving normal or nearly normal skin appearance. Subjects report little or no discomfort after treatment, and the treatment is tolerable with simply topical anesthesia. Moreover, this treatment is fast, completely non-invasive, tolerable, and is the only one that shows selectivity for the tumors. As a dermatologist who treats patients both in the office and in operating rooms, I am sure that this treatment can be done in the office rather than in an operating room. It takes about 10 seconds to position the laser and treat a tumor, implying that a hundred tumors could be treated in about 15 minutes. There is no open wound; sutures or antibiotics are not needed, and activity after treatment is not restricted.

Negative pressure-assisted alexandrite laser treatment of cNF tumors is a practical, effective, safe, tumor-selective way of clearing cNF tumors in patients with NF-1. One non-limiting example laser involved is popular and available in dermatology (it is widely used for laser hair removal). Alexandrite is not the only laser to be used with this invention. Other vascular targeting lasers include Nd: YAG, diode, Nd and other fiber lasers. Wavelength-filtered xenon flashlamp sources (IPLs) could be used as well. Whatever light source is used, the wavelength emitted should be absorbed by hemoglobins, and should penetrate at least to the depth of the target vessels. Most cNF tumors are 1-4 mm in diameter at the time they become visible; the wavelength region capable of penetration over this distance is approximately 530-1100 nm. The alexandrite laser wavelength at approximately 760 nm coincides with an absorption band of deoxyhemoglobin, and penetrates well through the entire skin.

The negative-pressure chamber can also be breathable, i.e., the negative-pressure chamber could have flow through it of air or other gas, and yet still be at a pressure below one atmosphere, i.e., a negative pressure. In one prototype of the invention used in a human clinical trial of neurofibromatosis treatment, I had small holes drilled in the negative-pressure chamber to admit some air flow, and yet maintain a negative pressure in the chamber (of about 400 mmHg, about half an atmosphere). This was done because when cryogen spray cooling of the skin surface was added, there was excess evaporated cryogen gas filling the negative-pressure chamber. It turns out that some cryogen vapors may be flammable and the problem was solved by letting enough air flow through the negative-pressure chamber to clear the cryogen vapor before launching a laser pulse.

Another relevant application for this invention is treatment of basal cell carcinoma (BCC), the most common cancer of mankind. Because this laser treatment is entirely non-invasive, very rapid compared with all other local destruction methods, and requires no surgical dressings or suture removal, physicians would favor this method over others for significant fraction, if not most of BCC. Still other common clinical applications include cutaneous lesions currently treated with vascular-targeted selective photothermolysis. Removal of warts, especially periungual and plantar warts, is another common problem for which I expect this invention will work well.

A pressure gradient within the dermis is necessary to draw blood into the target lesion, and that pressure gradient must be applied long enough to allow blood flow and engorgement of the target vessels. Prior devices force the skin against a flat window, defeating the pressure gradient within dermis. In prior devices, negative pressure is also applied for a very brief time, insufficient to allow vessel engorgement prior to laser treatment. In contrast, in this invention blood flow is specifically allowed to enter and engorge target blood vessels.

### Methods

I compared safety, tolerance and response of small (2-4 mm) tumors in 40 adult participants with NF1, for 12 months following a single treatment with four modalities: (1) insulated 21 gauge radiofrequency needle coagulation; (2) alexandrite laser with 755 nm wavelength, radiant exposure of 100 J/cm², 3 millisecond laser pulse, 8 mm laser spot size with negative pressure; (3) 980 nm diode laser delivered via 8 mm sapphire skin-contact window; and (4) intra- or injection of 10 mg/mL deoxycholic acid (Kybella®) volume approximately equal to that of the tumor. Topical anesthetic (5% lidocaine/prilocaine) was applied for 40 minutes before treatment of up to 10 cNF per modality, and up to four modalities in each participant. Pain, inflammation, pigmentation, wounding and adverse events were assessed. Three dimensional (Cherry Imaging) and standard clinical photographs were taken; biopsies were taken at 3 months for light microscopy.

### Results

Of 18 participants enrolled (ages 30-70, mean 47; 5M/8F), after seven had completed the 3 month assessments; 227 cNF tumors had been treated. No adverse events >grade 2 occurred. All modalities reduced or eliminated some cNF by 3 months post-treatment, with large variation between tumors and between participants. Strong pain limited the 980 nm laser treatment dose, which produced no apparent tumor reduction in most participants. Alexandrite laser was better tolerated, caused tumor-selective immediate purpura, and in some participants complete tumor clearance with normal skin appearance. Deoxycholate injection was nearly painless; its cNF reduction ranged from none to nearly complete. Pain during insulated-needle radiofrequency coagulation ranged from intolerable to nil, and tumor reduction appeared to correspond with the zone of coagulation. No cNF stimulation or recurrence was seen.

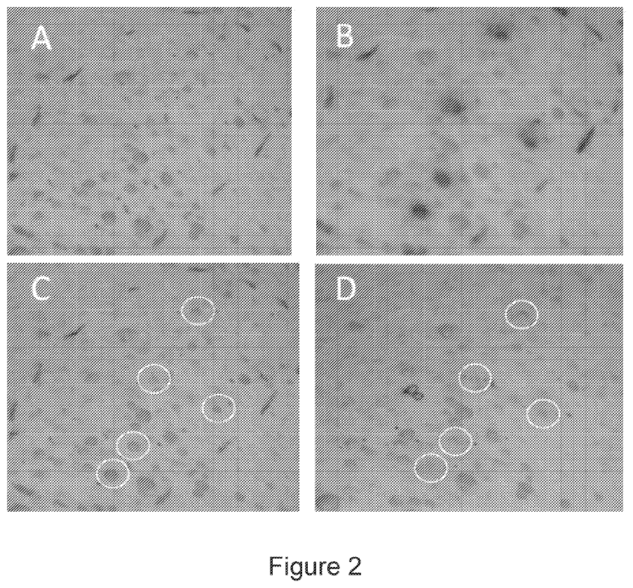

FIG. 2 shows neurofibroma tumor response to treatment using a non-limiting example device of the present disclosure, in a person with neurofibromatosis type I (NF-1). Panel (A) shows a skin region prior to treatment. Panel (B) shows immediate response after treatment with a negative-pressure assisted alexandrite laser at 755 nm. 3 milliseconds, 100 J/cm², 8 mm spot size exposure, showing darkening due to photocoagulation of tumor target blood vessels; treatment of all five tumors took approximately 1 minute. Panel (C) shows the pre-treatment photo with the five treated tumors marked (white circles). Panel (D) shows elimination (in two) and substantial reduction (in three) of all treated tumors, 3 months after treatment, without scarring or other side effects.

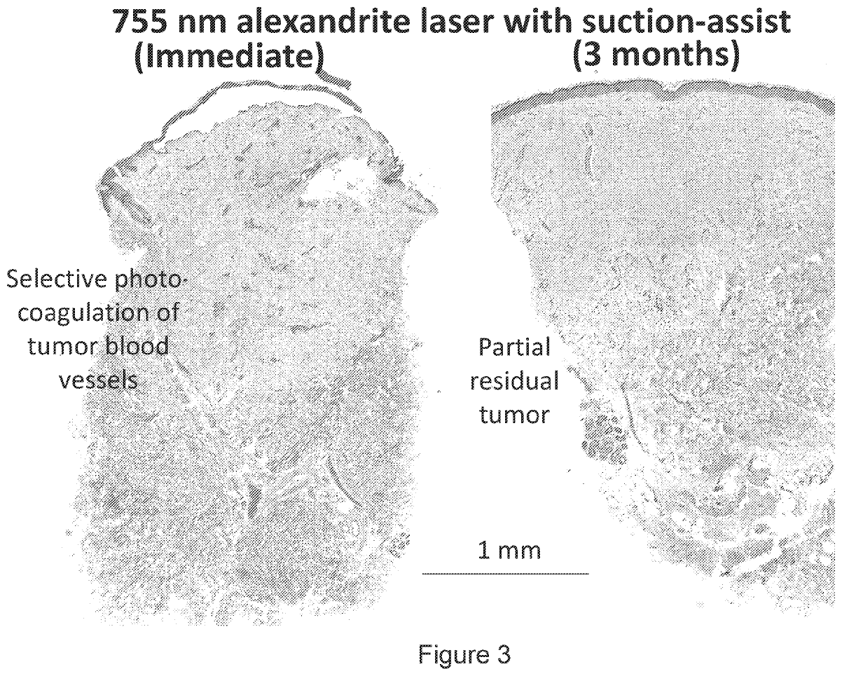

I biopsied cNF lesions after treatment this way, and the blood vessels are clearly distended (by suction) and then coagulated due to selective heating by the alexandrite laser pulse. See FIG. 3 which shows a microscopy (H&E stain) of a human neurofibroma tumor after treatment with a prototype negative-pressure device and alexandrite laser system. A fluence of 100 J/cm² was used, with an 8 mm diameter exposure spot at 755 nm wavelength, and 3 ms pulse duration, with a pressure of ˜125 mmHg (about ⅙ of an atmosphere, a negative pressure of about ⅚ atmosphere) within the negative pressure chamber. Immediately after treatment, throughout the tumor blood vessels are distended and thermally damaged, filled with non-flowing thermally-damaged blood. Three months after treatment, there is loss of the tumor and in the example shown in Figure, a small residual tumor remains after one treatment.

### CONCLUSIONS

All four modalities were safe and at least partially effective, with important practical differences. The 980 nm laser treatment was too painful and had low efficacy. In contrast, alexandrite laser was better tolerated, rapid (5-10 tumors/minute), tumor-selective and produced complete tumor clearance (gross and microscopic) in some participants. Deoxycholate injection, which dissolves cell membranes, was unexpectedly well tolerated and requires no technology. However, it and radiofrequency needle coagulation both require skillful placement into the cNF, and leave a minor open wound. I concluded that clearance of small cNF after one noninvasive local treatment according to the invention is possible.

For this Example, a prototype was made, and demonstrated to work well in an ongoing clinical trial. This Example compares response of cNF to other modalities including other lasers, radiofrequency surgery, and injection of a natural surfactant (deoxycholate). This invention, which in one non-limiting example embodiment of this Example used an alexandrite laser emitting 100 J/cm² fluence in an 8 mm diameter exposure spot at 755 nm wavelength, provides devices and methods for treating skin neoplasms (such as cutaneous neurofibroma tumors). The exposure parameters of the non-limiting example embodiment of this Example are exemplary, not limiting.

Based on this Example, a negative pressure handpiece system should include a sealed chamber contacting the skin, and a pump or other source for suction. The system may also include a pressure regulator and/or pressure gauge (as present in the prototype of this Example). Direct visualization of the skin to be treated is strongly preferred, so the sealed chamber can be transparent. Because of patient contact, that component may be disposable and/or sterilized. A laser or IPL device need be only slightly modified to accommodate this invention.

The most appropriate lasers for the invention are those at wavelengths with effective absorption in blood, at various penetration depths, and with appropriate fluence and pulse duration to cause irreversible selective vascular damage. These include alexandrite, pulsed dye, KTP, Nd: YAG, and various fiber and diode lasers. These are widely available from various biomedical laser companies.

Thus, the present invention provides devices and methods for treating skin neoplasms (such as cutaneous neurofibroma tumors) and skin malformations of a subject. The invention provides an essential new capability for treating NF and other skin conditions.

In light of the principles and example embodiments described and illustrated herein, it will be recognized that the example embodiments can be modified in arrangement and detail without departing from such principles. Also, the foregoing discussion has focused on particular embodiments, but other configurations are also contemplated. In particular, even though expressions such as “in one embodiment”, “in another embodiment”, “in other embodiments”, “in some embodiments”, or the like are used herein, these phrases are meant to generally reference embodiment possibilities, and are not intended to limit the invention to particular embodiment configurations. As used herein, these terms may reference the same or different embodiments that are combinable into other embodiments. As a rule, any embodiment referenced herein is freely combinable with any one or more of the other embodiments referenced herein, and any number of features of different embodiments are combinable with one another.

Although the invention has been described in considerable detail with reference to certain embodiments, one skilled in the art will appreciate that the present invention can be used in alternative embodiments to those described, which have been presented for purposes of illustration and not of limitation. Therefore, the scope of the appended claims should not be limited to the description of the embodiments contained herein.

1. A device for treating a condition of skin of a subject, the device comprising:
a chamber having an end wall, a side wall extending away from the end wall, and an opening opposite the end wall, wherein the end wall, the side wall and the opening define an interior space of the chamber, wherein the end wall comprises a transparent window, wherein the side wall terminates in a contact surface for sealing against skin of a subject, wherein the contact surface defines the opening, wherein the side wall has a longitudinal length such that the end wall is spaced greater than ten millimeters from the skin of the subject when the contact surface seals against skin of the subject;
a vacuum source in fluid communication with the interior space of the chamber, the vacuum source being structured to create negative pressure in the chamber; and
a light source for irradiating a region of the skin of the subject with light, the light source being selected from the group consisting of laser light sources and wavelength-filtered light sources, the light source being positioned to direct the light through the transparent window at the region of the skin of the subject when the light source is activated.

2. The device of claim 1 further comprising:
a pressure regulator operable to control a suction level of the vacuum source such that when the contact surface seals against the skin of the subject, the negative pressure created in the chamber by the vacuum source does not draw the skin of the subject into contact with the end wall of the chamber.

3. The device of claim 1 wherein:
the light source emits wavelengths that are preferentially absorbed by hemoglobin and/or oxyhemoglobin.

4. The device of claim 1 wherein:
the light source emits wavelengths in a range of wavelengths from 500 nanometers to 2000 nanometers.

5. (canceled)

6. (canceled)

7. The device of claim 1 wherein:
the light source is a laser light source.

8. The device of claim 1 wherein:
the light source is an intense-pulsed-light source.

9. The device of claim 1 wherein:
the light source is a wavelength-filtered light source.

10. The device of claim 1 wherein:
a spot size of the light has a diameter in a range of 1 to 20 millimeters.

11. The device of claim 1 wherein:
the negative pressure is between about −10 and −760 mm Hg.

12. The device of claim 1 wherein: the end wall and the side wall of the chamber are transparent.

13. (canceled)

14. (canceled)

15. (canceled)

16. (canceled)

17. The device of claim 1 further comprising:
a coolant source in fluid communication with the interior space of the chamber.

18. The device of claim 1 further comprising:
a controller in electrical communication with the vacuum source and the light source, the controller being configured to execute a program stored in the controller to:
(i) activate the vacuum source to create the negative pressure in the chamber, and
(ii) irradiate the region of the skin with light for a period of time such that the light is provided at a radiant exposure of at least 5 J/cm².

19. The device of claim 18 further comprising:
a pressure sensor for determining a pressure level within the interior space of the chamber, the pressure sensor being in electrical communication the controller,
wherein the controller executes the program stored in the controller to:
(iii) receive electrical signals from the pressure sensor and continue activation of the vacuum source for a time duration based on electrical signals received from the pressure sensor.

20. (canceled)

21. The device of claim 2 further comprising:
a controller in electrical communication with the pressure regulator and the light source, the controller being configured to execute a program stored in the controller to:
(i) activate the pressure regulator to create the negative pressure in the chamber, and
(ii) irradiate the region of the skin with light for a period of time such that the light is provided at a radiant exposure of at least 5 J/cm².

22. The device of claim 21 further comprising:
a pressure sensor for determining a pressure level within the interior space of the chamber, the pressure sensor being in electrical communication the controller,
wherein the controller executes the program stored in the controller to:
(iii) receive electrical signals from the pressure sensor and continue activation of the pressure regulator for a time duration based on electrical signals received from the pressure sensor.

23. (canceled)

24. The device of claim 18 wherein:
the controller executes the program stored in the controller to:
(ii) irradiate the region of the skin with light for the period of time such that the light is provided at a radiant exposure of at least 50 J/cm².

25. (canceled)

26. The device of claim 18 wherein:
the controller executes the program stored in the controller to:
(ii) irradiate the region of the skin with pulsed light at a pulse duration of 1 to 10 milliseconds.

27. The device of claim 19 wherein:
the time duration is greater than 1 second.

28. (canceled)

29. (canceled)

30. (canceled)

31. (canceled)

32. A method for treatment of a skin neoplasm of skin of a subject, the method comprising:
(a) providing a chamber having an end wall, a side wall extending away from the end wall, and an opening opposite the end wall, wherein the end wall, the side wall and the opening define an interior space of the chamber, wherein the side wall terminates in a contact surface for sealing against skin of a subject, wherein the contact surface defines the opening;
(b) positioning the contact surface of the chamber on the skin of the subject such that the contact surface surrounds the skin neoplasm and the contact surface seals against the skin of the subject;
(c) creating negative pressure in the interior space of the chamber; and
(d) irradiating the skin neoplasm with light from a light source selected from the group consisting of laser light sources and wavelength-filtered light sources.

33. (canceled)

34. (canceled)

35. (canceled)

36. (canceled)

37. (canceled)

38. A method for treatment of a skin malformation of skin of a subject, the method comprising:
(a) providing a chamber having an end wall, a side wall extending away from the end wall, and an opening opposite the end wall, wherein the end wall, the side wall and the opening define an interior space of the chamber, wherein the side wall terminates in a contact surface for sealing against skin of a subject, wherein the contact surface defines the opening;
(b) positioning the contact surface of the chamber on the skin of the subject such that the contact surface surrounds the skin malformation and the contact surface seals against the skin of the subject;
(c) creating negative pressure in the interior space of the chamber; and
(d) irradiating the skin malformation with light from a light source selected from the group consisting of laser light sources and wavelength-filtered light sources.

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)
